SEQUENCE LISTING (15 sequences)

SEQ ID NO: 1 - DNA, 16 bp, synthetic construct
  source 1..16: mol_type = other DNA
  misc_feature 1..16: note = 5' constant region of ssDNA in ssDNA library
ggcaggaagacaaaca

SEQ ID NO: 2 - DNA, 16 bp, synthetic construct
  source 1..16: mol_type = other DNA
  misc_feature 1..16: note = 3' constant region of ssDNA in ssDNA library
tggtctgtggtgctgt

SEQ ID NO: 3 - DNA, 34 bp, synthetic construct
  source 1..34: mol_type = other DNA
  misc_feature 1..34: note = polyGC fragments fixed on beads
ttttttttttgcgcgcgcgcgcgcgcgcgcgcgc

SEQ ID NO: 4 - DNA, 16 bp, synthetic construct
  source 1..16: mol_type = other DNA
  misc_feature 1..16: note = forward primer for amplification of ssDNA
ggcaggaagacaaaca

SEQ ID NO: 5 - DNA, 16 bp, synthetic construct
  source 1..16: mol_type = other DNA
  misc_feature 1..16: note = reverse primer for amplification of ssDNA
acagcaccacagacca

SEQ ID NO: 6 - DNA, 74 bp, synthetic construct
  source 1..74: mol_type = other DNA
  misc_feature 1..74: note = DNA aptamer MeLW1 specifically binding to methylated DNA fragments
ggcaggaagacaaacaccgtggtctgtggtgctgtacgtggcaggaagacaaacaccgtggtctgtggtgctgt

SEQ ID NO: 7 - DNA, 42 bp, synthetic construct
  source 1..42: mol_type = other DNA
  misc_feature 1..42: note = core region in DNA aptamer MeLW1
ccgtggtctgtggtgctgtacgtggcaggaagacaaacaccg

SEQ ID NO: 8 - DNA, 72 bp, synthetic construct
  source 1..72: mol_type = other DNA
  misc_feature 1..72: note = DNA aptamer MeLW2 specifically binding to methylated DNA fragments
ggcaggaagacaaacaccatagccgccccacatccacacatccattagtcatgtcgtggtctgtggtgctgt

SEQ ID NO: 9 - DNA, 40 bp, synthetic construct
  source 1..40: mol_type = other DNA
  misc_feature 1..40: note = core region in DNA aptamer MeLW2
ccatagccgccccacatccacacatccattagtcatgtcg

SEQ ID NO: 10 - DNA, 20 bp, synthetic construct
  source 1..20: mol_type = other DNA
  misc_feature 1..20: note = forward primer for amplification of BRCA1 promoter fragment
actgcgactgcgcggcgtga

SEQ ID NO: 11 - DNA, 20 bp, synthetic construct
  source 1..20: mol_type = other DNA
  misc_feature 1..20: note = reverse primer for amplification of BRCA1 promoter fragment
gggcccagttatctgagaaa

SEQ ID NO: 12 - DNA, 17 bp, synthetic construct
  source 1..17: mol_type = other DNA
  misc_feature 1..17: note = forward primer for amplification of BRCA2 promoter fragment
cccctcacgcttctccc

SEQ ID NO: 13 - DNA, 16 bp, synthetic construct
  source 1..16: mol_type = other DNA
  misc_feature 1..16: note = reverse primer for amplification of BRCA2 promoter fragment
gacggttgggatgcct

SEQ ID NO: 14 - DNA, 18 bp, synthetic construct
  source 1..18: mol_type = other DNA
  misc_feature 1..18: note = forward primer for amplification of BRCA1 promoter fragment
gcagactgggtggccaat

SEQ ID NO: 15 - DNA, 20 bp, synthetic construct
  source 1..20: mol_type = other DNA
  misc_feature 1..20: note = reverse primer for amplification of BRCA1 promoter fragment
aagcagcagcctctcagaat

(19) United States
(12) Patent Application Publication
(10) Pub. No.: US 2024/0218375 A1
(43) Pub. Date: Jul. 4, 2024

(54) DNA APTAMER SPECIFICALLY BINDING TO METHYLATED DNA FRAGMENT AND SELECTION METHOD AND USE THEREOF

(71) Applicant: National Tsing Hua University, Hsinchu City (TW)

(72) Inventors: Gwo-Bin LEE, Hsinchu City (TW); Chih-Hung WANG, Hsinchu City (TW)

(21) Appl. No.: 18/298,734

(22) Filed: Apr. 11, 2023

(30) Foreign Application Priority Data
Dec. 29, 2022 (TW) 111150673

(51) Int. Cl. C12N 15/115; C12Q 1/6897

(52) U.S. Cl. CPC C12N 15/115; C12Q 1/6897; C12N 2310/16; C12N 2320/11; C12Q 2600/154

(57) ABSTRACT

A DNA aptamer specifically binding to a methylated DNA fragment includes a nucleotide sequence selected from the group consisting of SEQ ID NO:7 and SEQ ID NO:9. In addition, a method for detecting a methylated DNA fragment in a sample and a method for selecting a DNA aptamer specifically binding to the methylated DNA fragment are also disclosed.

### CROSS-REFERENCE TO RELATED APPLICATION

This application claims priority to Taiwanese Invention Patent Application No. 111150673, filed on Dec. 29, 2022.

### REFERENCE TO SEQUENCE LISTING, TABLE OR COMPUTER PROGRAM

The Sequence Listing submitted concurrently herewith with a file name of “NPE-29147-AM-Sequence11.xml,” a creation date of Mar. 15, 2023, and a size of 19.7 kilobytes, is part of the specification and is incorporated by reference herein.

### FIELD

The present disclosure relates to a DNA aptamer specifically binding to a methylated DNA fragment and a method for selecting the same. The present disclosure also relates to a method for detecting a methylated DNA fragment in a sample.

### BACKGROUND

DNA methylation is a typical form of epigenetic regulation in organisms, which regulates the expression of a gene by adding a methyl group to a DNA molecule (e.g., a CpG island located in a promoter region). Many diseases have been found to be related to abnormal DNA methylation. For example, aberrant promoter hypermethylation of tumor suppressor genes is known to be a crucial factor involved in carcinogenesis. Accordingly, rapid detection of a methylated DNA fragment in a sample (such as a blood sample) would facilitate the study of pathogenic mechanisms, as well as therapeutic targets.

Methylated DNA immunoprecipitation (MeDIP) is a versatile technique in large-scale purification of methylated DNA fragments, and utilizes a monoclonal antibody raised against 5-methylcytosine to capture various methylated DNA fragments in a sample. The captured methylated DNA fragments can be subjected to molecular techniques (such as DNA amplification, microarray analysis, and next-generation sequencing) so as to detect the gene of interest. However, MeDIP has the problems of high cost and non-specific binding due to use of antibodies.

A DNA aptamer is a short single-stranded DNA (ssDNA), which can specifically bind to a certain target molecule (e.g., a heavy metal, a protein, and a cell) due to its tertiary structure, and can be selected from a library of ssDNA using systematic evolution of ligands by exponential enrichment (SELEX) technology.

As far as the applicant is aware, there have been no publications or prior art documents which disclose that a DNA aptamer can be used to capture or detect a methylated DNA fragment.

### SUMMARY

Accordingly, in a first aspect, the present disclosure provides a DNA aptamer specifically binding to a methylated DNA fragment, which can alleviate at least one of the drawbacks of the prior art. The DNA aptamer specifically binding to the methylated DNA fragment includes a nucleotide sequence selected from the group consisting of SEQ ID NO:7 and SEQ ID NO:9.

In a second aspect, the present disclosure provides a method for detecting a methylated DNA fragment in a sample, which can alleviate at least one of the drawbacks of the prior art. The method includes:
mixing the aforesaid DNA aptamer with the sample, so that the DNA aptamer binds to the methylated DNA fragment in the sample to form a DNA aptamer-methylated DNA fragment complex; and
detecting the DNA aptamer-methylated DNA fragment complex.

In a third aspect, the present disclosure provides a method for selecting a DNA aptamer specifically binding to a methylated DNA fragment, which can alleviate at least one of the drawbacks of the prior art. The method includes:
providing a library of candidate DNA aptamers;
incubating the library of candidate DNA aptamers with the methylated DNA fragment attached to a solid support at a temperature ranging from 25° C. to 37° C. and under at least one of the following conditions: a) in the presence of phosphate-buffered saline having a pH value ranging from pH 5.5 to pH 7.4; b) in the presence of 10 mM to 50 mM of lactic acid; and c) in the presence of blood, so that a DNA aptamer binds to the methylated DNA fragment; and
amplifying the DNA aptamer bound to the methylated DNA fragment.

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The terms “nucleic acid”, “nucleic acid sequence”, and “nucleic acid fragment”, as used herein, refer to a deoxyribonucleotide or ribonucleotide sequence in single-stranded or double-stranded form, which includes naturally occurring nucleotides or artificial chemical mimics. The term “nucleic acid”, as used herein, is interchangeable with the terms “gene”, “DNA”, “cDNA”, “mRNA”, “oligonucleotide”, and “polynucleotide” in use.

As used herein, the terms “nucleic acid fragment” and “DNA fragment” can be interchangeably used, and refer to a DNA polymer, in the form of a separate segment or as a component of a larger DNA construct, which has been derived either from isolated DNA or which is synthesized chemically or enzymatically such as by methods disclosed elsewhere.

Unless otherwise indicated, a nucleic acid sequence, in addition to the specific sequences described herein, also covers its complementary sequence, and the conservative analogs, related naturally occurring structural variants and/or synthetic non-naturally occurring analogs thereof.

Unless otherwise indicated, whenever a nucleic acid sequence is represented, it will be understood that the nucleotides are in 5′ to 3′ order from left to right and that “A” denotes deoxyadenosine or an analog thereof, “C” denotes deoxycytidine or an analog thereof, “G” denotes deoxyguanosine or an analog thereof, and “T” denotes thymidine or an analog thereof.

The term “promoter”, as used herein, refers to a DNA sequence, which is generally located upstream of a gene present in a DNA polymer, and provides a site for initiation of the transcription of the gene into mRNA.

The term “DNA aptamer”, as used herein, refers to a short single-stranded DNA (DNAss) with a distinct tertiary structure, which exhibits high specificity to target molecules.

By conducting research, the applicant surprisingly found that a DNA aptamer including a nucleotide sequence of SEQ ID NO:7 or a nucleotide sequence of SEQ ID NO:9 can exhibit high specificity and sensitivity on the detection of a methylated DNA fragment, and hence can be utilized to capture a variety of methylated DNA fragments in a sample. The thus captured methylated DNA fragments can be further subjected to a DNA amplification reaction, so as to determine whether a methylated target DNA fragment (such as a promoter of a tumor suppressor gene) is present in the sample.

Accordingly, the present disclosure provides a DNA aptamer specifically binding to a methylated DNA fragment, which includes a nucleotide sequence selected from the group consisting of SEQ ID NO:7 and SEQ ID NO:9. In certain embodiments, the DNA aptamer includes a nucleotide sequence selected from the group consisting of SEQ ID NO:6 and SEQ ID NO:8. In particular, SEQ ID No:6 includes a core region having a nucleotide sequence of SEQ ID NO:7, and SEQ ID No:8 includes a core region having a nucleotide sequence of SEQ ID NO:9.

According to the present disclosure, the DNA aptamer can be attached to a solid support utilizing techniques well known to those skilled in the art, so that the methylated DNA fragment to be specifically bound by the DNA aptamer can be captured. Examples of the solid support suitable for use in the present disclosure may include, but are not limited to, a plate, a tube, a membrane, a resin, and a bead. In certain embodiments, the solid support may be a bead selected from the group consisting of a magnetic bead, an agarose bead, a Sephadex® bead, a glass bead, and combinations thereof. In an exemplary embodiment, the solid support is a magnetic bead having a carboxylic acid group.

The present disclosure also provides a method for detecting a methylated DNA fragment in a sample, which includes:
mixing the aforesaid DNA aptamer with the sample, so that the DNA aptamer binds to the methylated DNA fragment in the sample to form a DNA aptamer-methylated DNA fragment complex; and
detecting the DNA aptamer-methylated DNA fragment complex.

According to the present disclosure, the methylated DNA fragment may be derived from any gene of interest so as to analyze the epigenetic regulation of the interested gene in the sample. In certain embodiments, the methylated DNA fragment is derived from a promoter of a tumor suppressor gene selected from the group consisting of a promoter of breast cancer gene 1 (BRCA1), a promoter of breast cancer gene 2 (BRCA2), a promoter of Ras association domain family 1A (RASSF1A) gene, a promoter of opioid binding protein/cell adhesion molecule-like (OPCML) protein gene, a promoter of p16INK4α gene, a promoter of Kruppel-like transcription factor 11 (KLF11) gene, a promoter of ADP-ribosylhydrolase (ADPRH) gene, a promoter of globoside alpha-1,3-N-acetylgalactosaminyltransferase 1 (GBGT1) gene, a promoter of PDZ and LIM domain protein 2 (PDLIM2) gene, and combinations thereof. In an exemplary embodiment, the promoter of the tumor suppressor gene is selected from the group consisting of a promoter of BRCA1 and a promoter of BRCA2.

According to the present disclosure, the sample may be a biological sample. In certain embodiments, the sample is a biological simple obtained from a subject having or suspected of having a cancer. As used herein, the “biological sample” is a sample obtained from an organism (e.g., an animal subject) or from components (e.g., cells and tissues) of the organism. Examples of the biological sample include, but are not limited to, a blood sample, a plasma sample, a serum sample, a tear sample, a saliva sample, a cerebrospinal fluid sample, a feces sample, a tissue biopsy, a urine sample, and combinations thereof.

According to the present disclosure, before mixing the sample with the DNA aptamer, the sample may be subjected to a pretreatment selected from the group consisting of a genomic DNA extraction treatment, a DNA fragmentation treatment, and a combination thereof. The procedures and conditions for extracting genomic DNA are within the expertise and routine skills of those skilled in the art.

According to the present disclosure, a detectable label may be attached or conjugated to the DNA aptamer using techniques well known to those skilled in the art, so as to detect the DNA aptamer-methylated DNA fragment complex. Examples of the detectable label suitable for use in the disclosure include, but are not limited to, a hapten label, such as biotin and digoxigenin (Dig); a chemiluminescent label; a fluorescent label, such as fluorescein, rhodamine, FAM, TET, HEX, JOE, TAMA, NTB, TAMRA, ROX, VIC, NED, Yakima Yellow, BHQ-1, BHQ-2, BHQ-3, Iowa Black FQ, Iowa Black RQ, DABCYL, DABSYL, ElleQuencher, Eclipse Dark Quencher, Methyl Red, Texas Red, Malachite Green, Disperse Blue 3, BODIPY 493/503, a cyanin (Cy) (e.g., Cy2, Cy3, Cy3.5, Cy5, Cy5.5, and Cy7), an Alexa Fluor (e.g., Alexa Fluor 488, 532, 546, 555, 568, 594, 610, 647, and 680), a PromoFluor (e.g., PromoFluor 488, 555, 590, 633, 647, and 680), and an LC-Red (e.g., LC-Red 610, 640, 670 and 705); an enzyme label; a radioactive label such as ³²P; a particle label, such as gold colloids and quantum dots; and a colorimetric label, such as colored glass beads and plastic beads.

According to the present disclosure, the DNA aptamer-methylated DNA fragment complex may be detected by subjecting the DNA aptamer-methylated DNA fragment complex to a DNA amplification reaction using a primer pair which is designed to target a distinct region of a gene of interest. In certain embodiments, the primer pair is designed to target a distinct region of a promoter of a tumor suppressor gene described above.

According to the present disclosure, the DNA amplification reaction may be carried out using at least one of the following methodologies: polymerase chain reaction (PCR), real-time quantitative PCR, nested PCR, hot-start PCR, in-situ PCR, micro PCR, and multiplex PCR. The procedures and conditions for extracting genomic DNA are within the expertise and routine skills of those skilled in the art.

According to the present disclosure, the DNA amplification reaction may be carried out on a biochip selected from the group consisting of a microfluidic chip (e.g., an integrated microfluidic chip, a reaction chamber chip, and an analysis chip) and a lab-on-a-chip.

Moreover, the present disclosure provides a method for selecting a DNA aptamer specifically binding to a methylated DNA fragment, which includes:
providing a library of candidate DNA aptamers;
incubating the library of candidate DNA aptamers with the methylated DNA fragment attached to a solid support at a temperature ranging from 25° C. to 37° C. and under at least one of the following conditions: a) in the presence of phosphate-buffered saline having a pH value ranging from pH 5.5 to pH 7.4, b) in the presence of 10 mM to 50 mM of lactic acid, and c) in the presence of blood, so that a DNA aptamer binds to the methylated DNA fragment; and
amplifying the DNA aptamer bound to the methylated DNA fragment.

According to the present disclosure, the library of candidate DNA aptamers may be a single-stranded DNA library that can be used in a process which combines combinatorial chemistry with in vitro evolution, commonly known as systematic evolution of ligands by exponential enrichment (SELEX). In certain embodiments, each candidate DNA aptamer is a single-stranded DNA having 20 to 80 random nucleotides. In an exemplary embodiment, each candidate DNA aptamer is a single-stranded DNA having 30 to 50 random nucleotides.

According to the present disclosure, the methylated DNA fragment may be attached to the solid support using techniques well known to those skilled in the art, so that the DNA aptamer to be specifically binding to the methylated DNA fragment can be captured.

According to the present disclosure, the amplified DNA aptamers may be further subjected to a negative selection process. Therefore, the method may further include:
incubating the amplified DNA aptamers with unmethylated DNA fragments each of which is attached to a solid support, so that one part of the amplified DNA aptamers binds to the unmethylated DNA fragments and the other part of the amplified DNA aptamers does not bind to the unmethylated DNA fragments;
removing the part of the amplified DNA aptamers which binds to the unmethylated DNA fragments; and
amplifying the part of the amplified DNA aptamers not binding to the unmethylated DNA fragments.

In certain embodiments, the DNA aptamer selected according to the method of the present disclosure has a nucleotide sequence selected from the group consisting of SEQ ID NO:7 and SEQ ID NO:9. In an exemplary embodiment, the DNA aptamer has a nucleotide sequence selected from the group consisting of SEQ ID NO:6 and SEQ ID NO:8. In particular, SEQ ID NO:6 includes a core region having a nucleotide sequence of SEQ ID NO:7, and SEQ ID NO:8 includes a core region having a nucleotide sequence of SEQ ID NO:9.

The disclosure will be further described by way of the following examples. However, it should be understood that the following examples are solely intended for the purpose of illustration and should not be construed as limiting the disclosure in practice.

### EXAMPLES

### General Experimental Materials

### 1. Genomic DNA Fragments of Cancer Cell Lines:

The genomic DNA fragments of a respective one of the cancer cell lines shown in Table 1 were obtained by subjecting the cancer cells to genomic DNA extraction using QIAamp DNA Mini Kit (Qiagen, Cat. No. 51306) according to the manufacturer's instructions, followed by sonication.

TABLE 1

| Cancer | Cell line | Source |
|---|---|---|
| Ovarian cancer | Human ovarian cancer cell | National Cheng Kung |
| | line A2780 | University |
| | Human ovarian cancer cell | |
| | line OVCAR-8 | |
| Cervical cancer | Human cervical cancer cell | Chung Shan Medical |
| | line HeLa | University |
| Triple negative | Human breast cancer cell | |
| breast cancer | line MDA-MB-231 | |
| Lung cancer | Human lung adenocarcinoma | |
| | cell line A549 | |

### Example 1. Selection of DNA Aptamers Specifically Binding to Methylated DNA Fragments

### Experimental Materials

### 1. Library of Candidate DNA Aptamers

The library of candidate DNA aptamers used in this example was synthesized by Boston Biotechnology Co., Ltd. Each of the candidate DNA aptamers includes a 5′ conserved region having 16 nucleotides (nt) (SEQ ID NO:1), a core region (N) having approximately 40 random nucleotides, and a 3′ conserved region having 16 nt (SEQ ID NO:2) in a direction from 5′-end to 3′-end as shown below:

5′-ggcaggaagacaaaca-N-tggtctgtggtgctgt-3′
(5′ conserved region) (3′ conserved region)

### 2. Methylated and Unmethylated Poly-GC Fragments

The methylated and unmethylated poly-GC fragments used in this example were synthesized by Boston Biotechnology Co., Ltd. Each of the methylated and the unmethylated poly-GC fragments includes a poly-T region of 10 nt and a poly-GC region of 24 nt in a direction from 5′-end to 3′-end as shown below:

(SEQ ID NO: 3)
5′-tttttttttt-gcgcgcgcgcgcgcgcgcgcgcgc-3′
(poly-T region) (poly-GC region)

A respective one of the methylated and unmethylated poly-GC fragments was immobilized on magnetic beads having carboxylic acid groups (Invitrogen Dynabeads™ MyOne™ Carboxylic Acid, Cat. No. 65011) through the poly-T region thereof using N-(3-dimethylaminopropyl)-N′-ethylcarbodiimide hydrochloride according to the method described in C. H. Wang et al., (2011), Biosensors and Bioelectronics, January 15; 26(5):2045-52.

The resultant methylated poly-GC fragment-conjugated magnetic beads and unmethylated poly-GC fragment-conjugated magnetic beads were used in the following experiment.

### Experimental Procedures

DNA aptamers specifically binding to methylated DNA fragments were selected by systematic evolution of ligands by exponential enrichment (SELEX) technology. Eight rounds of the SELEX process were performed using the operating conditions as shown in Table 2 below. Briefly, in the first round of SELEX, 10 μL of the library of the candidate DNA aptamers (100 μM), 10 μL of the methylated poly-GC fragment-conjugated magnetic beads (10⁵ beads/μL), and 80 μL of phosphate-buffered saline (PBS) (pH 7.4) were mixed, followed by incubation at 25° C. and 20 rpm for 30 minutes. Afterward, the target DNA aptamer-bound magnetic beads were collected using a magnetic stand (Invitrogen, Cat. No. 65011) and then washed with 200 μL of PBS (pH 7.4) twice, followed by suspending in 50 μL of deionized water so as to form a suspension. Next, 10 μL of the suspension was subjected to polymerase chain reaction (PCR) that was performed using the designed primer pair shown in Table 3 and the reaction conditions shown in Table 4, so that the target DNA aptamers bound to the methylated poly-GC fragments were amplified to obtain PCR products which served as a library of candidate DNA aptamers for the next round of SELEX.

TABLE 2

| | Poly-GC fragments immobilized | Incubation | Temper- |
|---|---|---|---|
| Round | on magnetic beads | condition | ature |
| 1^{st} | Methylated poly-GC fragments | PBS (pH 7.4) | 25° C. |
| 2^{nd} | Unmethylated poly-GC | PBS (pH 7.4) | 25° C. |
| | fragments | | |
| 3^{rd} | Methylated poly-GC fragments | PBS (pH 7.4) | 37° C. |
| 4^{th} | Methylated poly-GC fragments | Blood^{a} | 25° C. |
| 5^{th} | Methylated poly-GC fragments | PBS (pH 6.5)^{b} | 25° C. |
| 6^{th} | Methylated poly-GC fragments | PBS (pH 5.5)^{b} | 25° C. |
| 7^{th} | Methylated poly-GC fragments | 50 mM of lactic | 25° C. |
| | | acid^{c} | |
| 8^{th} | Methylated poly-GC fragments | 10 mM of lactic | 25° C. |
| | | acid^{c} | |

^{a}provided by Department of Obstetrics and Gynecology of National Cheng Kung University Hospital.
^{b}pH value was adjusted using 0.1N HCl and 1N NaOH.
^{c}prepared in PBS (pH 7.4).

TABLE 3

| | Corresponding | | Size of |
|---|---|---|---|
| | region in | Nucleotide | PCR |
| Primer | candidate | sequence | product |
| pair | DNA aptamers | (5′ → 3′) | (bp) |
| Forward | 5′ conserved | ggcaggaagacaaaca | 72 |
| primer | region | (SEQ ID NO: 4) | |
| Reverse | 3′ conserved | acagcaccacagacca | |
| primer | region | (SEQ ID NO: 5) | |

TABLE 4

| Contents | Volume (μL) |
|---|---|
| Library of candidate DNA aptamers | 10 |
| Forward primer (10 μM) | 0.3 |
| Reverse primer (10 μM) | 0.3 |
| 2X GoTaq ® Green Master Mix (Promega) | 12.5 |
| Sterile water | 1.9 |

Operation conditions: denaturation at 95° C. for 5 minutes, followed by 35 cycles of the following reactions: denaturation at 95° C. for 20 seconds, primer annealing at 56° C. for 15 seconds, extension at 72° C. for 15 seconds, and elongation at 72° C. for 10 minutes.

In the second round of SELEX, the PCR products obtained above served as the library of candidate DNA aptamers, and the unmethylated poly-GC fragment-conjugated magnetic beads were used in replacement of the foregoing methylated poly-GC fragment-conjugated magnetic beads. The procedures of the second round of SELEX were similar to those of the first round, except that the magnetic beads collected by the magnetic stand were discarded, and the remaining solution was collected, and then 10 μL of the remaining solution was subjected to PCR that was performed using the designed primer pair shown in Table 3 and the reaction conditions shown in Table 4, so that the target DNA aptamers not bound to the unmethylated poly-GC fragments were amplified to obtain PCR products which served as a library of candidate DNA aptamers for the next round of SELEX.

The incubation conditions and temperatures for the third to eighth rounds of SELEX were varied as shown in Table 2, and the procedures for the third to eight rounds of SELEX were similar to those of the first round. Thereafter, the PCR products obtained in the eighth round of SELEX were verified by sequencing analysis which was entrusted to Genomics BioSci & Tech Co., Ltd., so as to obtain DNA aptamers having different nucleotide sequences. A 74-nt aptamer named MeLW1 (SEQ ID No:6) and including a core region having a nucleotide sequence of SEQ ID NO:7, and a 72-nt aptamer named MeLW2 (SEQ ID NO.8) and including a core region having a nucleotide sequence of SEQ ID NO:9, were selected from the DNA aptamers thus obtained. A respective one of MeLW1 and MeLW2 was immobilized on magnetic beads using the method described in Section 2 of Experimental Materials in Example 1. The resultant MeLW1-conjugated magnetic beads and MeLW2-conjugated magnetic beads were used in the following experiments.

### Example 2. Evaluation for Detection Effect of Aforesaid Two DNA Aptamers on Methylated DNA Fragments

### Experimental Materials

### 1. Methylated and Unmethylated Fragments for BRCA1 and BRCA2 Promoters

Methylated and unmethylated fragments for BRCA1 promoter (corresponding to nucleotide residues 992 to 913 of the nucleotide sequence having NCBI Accession Number NG_056086.1) and BRCA2 promoter (corresponding to nucleotide residues 1139 to 885 of the nucleotide sequence having NCBI Accession Number NG_044973.1) used in this example were synthesized by Boston Biotechnology Co., Ltd.

### Experimental Procedures

### 1. Specificity Test

A respective one of the methylated BRCA1 promoter fragment, the unmethylated BRCA1 promoter fragment, the methylated BRCA2 promoter fragment, the unmethylated BRCA2 promoter fragment, and the genomic DNA fragments of human ovarian cancer cell line A2780, each having a volume of 10 μL and serving as a test sample, was mixed with 10 μL of MeLW1-conjugated magnetic beads (10⁵ beads/μL), followed by incubation at 25° C. and 20 rpm for 30 minutes. Afterward, the magnetic beads were collected using the magnetic stand and then washed with PBS, followed by suspending in 50 μL of deionized water so as to form a suspension. Next, 10 μL of the suspension was subjected to PCR that was performed using the designed primer pairs shown in Table 5 and the reaction conditions shown in Table 6, thereby obtaining a PCR product.

TABLE 5

| | | | Size of |
|---|---|---|---|
| | | | PCR |
| Target | Primer | Nucleotide | product |
| gene | pair | sequence | (bp) |
| BRCA1 | BRCA1F1 | actgcgactgcgcggcgtga | 79 |
| promoter | | (SEQ ID NO: 10) | |
| fragment | | | |
| | BRCA1R1 | gggcccagttatctgagaaa | |
| | | (SEQ ID NO: 11) | |
| BRCA2 | BRCA2F1 | cccctcacgcttctccc | 254 |
| promoter | | (SEQ ID NO: 12) | |
| fragment | | | |
| | BRCA2R1 | gacggttgggatgcct | |
| | | (SEQ ID NO: 13) | |

TABLE 6

| Contents | Volume (μL) |
|---|---|
| Fragments of BRCA1/BRCA2 promoter | 10 |
| Forward primer (10 μM) | 0.3 |
| Reverse primer (10 μM) | 0.3 |
| 2X GoTaq ® Green Master Mix (Promega) | 12.5 |
| Sterile water | 1.9 |

Operation conditions: denaturation at 95° C. for 5 minutes, followed by 35 cycles of the following reaction: denaturation at 95° C. for 20 seconds, primer annealing at 56° C. for 15 seconds, extension at 72° C. for 15 seconds, and elongation at 72° C. for 10 minutes.

The resultant PCR product was subjected to 2% agarose gel electrophoresis analysis for molecular weight verification.

In addition, the procedures for testing the specificity of MeLW2 were similar to those of MeLW1, except that the MeLW2-conjugated magnetic beads were used in replacement of the MeLW1-conjugated magnetic beads, and the primer pair BRCA1F2/BRCA1R2 as shown in Table 7 below was used for PCR, thereby obtaining a PCR product.

TABLE 7

| | | | Size of |
|---|---|---|---|
| | | | PCR |
| Target | Primer | Nucleotide | product |
| gene | pair | sequence | (bp) |
| BRCA1 | BRCA1F2 | gcagactgggtggccaat | 104 |
| promoter | | (SEQ ID NO: 14) | |
| fragment | BRCA1R2 | aagcagcagcctctcagaat | |
| | | (SEQ ID NO: 15) | |

The resultant PCR product was subjected to 2% agarose gel electrophoresis analysis for molecular weight verification.

### 2. Sensitivity Test

First, the genomic DNA fragments of human ovarian cancer cell line A2780 were subjected to 10-fold serial dilution, so as to obtain 3 dilutions having different concentrations (10 ng/μL, 1 ng/μL, and 0.1 ng/μL) and serving as test samples. Then, 10 μL of each diluted solutions was mixed with 10 μL of MeLW1-conjugated magnetic beads (10⁵ bead/μL), followed by incubation at 25° C. and 20 rpm for 30 minutes. Afterward, the magnetic beads were collected using the magnetic stand and then washed with PBS, followed by suspending in 50 μL of deionized water so as to form a suspension. Next, 10 μL of the suspension was subjected to PCR that was performed using the designed primer pair BRCA1F1/BRCA1R1 as shown in Table 5 and the reaction conditions shown in Table 6, thereby obtaining a PCR product.

The resultant PCR product was subjected to 2% agarose gel electrophoresis analysis for molecular weight verification.

### Results

### 1. Specificity Test

Based on the result of the agarose gel electrophoresis analysis (data not shown), it was found that by using the MeLW1-conjugated magnetic beads, a respective one of the methylated BRCA1 promoter fragment and the methylated BRCA2 promoter fragment was captured, and the PCR products having a size of approximately 79 bp and a size of approximately 254 bp were obtained using the primer pairs BRCA1F1/BRCA1 R1 and BRCA2F1/BRCA2R1, respectively. In addition, similar result was observed with respect to use of the genomic DNA fragments of human ovarian cancer cell line A2780, whereas no PCR product was obtained in regard to use of the unmethylated BRCA1 promoter fragment and the unmethylated BRCA2 promoter fragment.

Moreover, it was found that regarding the use of the MeLW2-conjugated magnetic beads, a respective one of the methylated BRCA1 promoter fragment and the methylated BRCA2 promoter fragment was capture, and the PCR products having a size of approximately 104 bp and a size of approximately 254 bp were obtained using the primer pairs BRCA1F2/BRCA1R and BRCA2F1/BRCA2R1, respectively. Additionally, similar result was observed with respect to use of the genomic DNA fragments of human ovarian cancer cell line A2780, whereas no PCR product was obtained in regard to use of the unmethylated BRCA1 promoter fragment and the unmethylated BRCA2 promoter fragment.

These results indicate that the two DNA aptamers (i.e., MeLW1 and MeLW2) of the present disclosure exhibit high specificity in detection of methylated DNA fragments.

### 2. Sensitivity Test

The result of the agarose gel electrophoresis analysis (data not shown) showed that the PCR product having a size of approximately 79 bp was obtained from both the diluted solution having the concentration of 10 ng/μL and the diluted solution having the concentration of 1 ng/μL, indicating that the limit of detection (LOD) of MeLW1 for methylated DNA fragments was 1 ng.

These results indicate that the DNA aptamer (i.e., MeLW1) of the present disclosure exhibit high sensitivity in detection of methylated DNA fragments.

### Example 3. Evaluation for Detection Effect of Aforesaid Two DNA Aptamers on Methylated DNA Fragments in Biological Samples

### 1. Detection Effect for Methylated DNA Fragments in Different Cancer Cells

The genomic DNA fragments of human ovarian cancer cell line OVCAR-8 (10 μL) was used to serve as a test sample, followed by conducting detection of methylated DNA fragments using MeLW1 and MeLW2 according to the method described in Section 1 of Experimental Procedures in Example 2.

The result of the agarose gel electrophoresis analysis (data not shown) showed that by using the MeLW1-conjugated magnetic beads and the MeLW2-conjugated magnetic beads, the methylated DNA regions of the genomic DNA fragments of human ovarian cancer cell line OVCAR-8 were captured, and PCR products having the predetermined sizes were obtained using the primer pairs BRCA1F1/BRCA1R1 and BRCA1F2/BRCA1 R2, respectively.

Additionally, the genomic DNA fragments of a respective one of human cervical cancer cell line HeLa, human breast cancer cell line MDA-MB-231, and human lung adenocarcinoma cell line A549 were subjected to the same analysis, and similar results were also observed.

These results indicate that MeLW1 and MeLW2 can effectively detect methylated DNA fragments in various cancer cells.

### 2. Detection Effect for Methylated DNA Fragments in a Blood Sample

First, 100 μL of a plasma sample obtained from a breast cancer patient was kindly provided by the Department of Obstetrics and Gynecology of National Cheng Kung University Hospital. Then, the plasma sample was subjected to detection of methylated DNA fragments using MeLW1 and MeLW2 according to the method described in Section 1 of Experimental Procedures in Example 2.

The results of the agarose gel electrophoresis analysis (data not shown) showed that by using the MeLW1-conjugated magnetic beads and the MeLW2-conjugated magnetic beads, the methylated DNA fragments in the plasma sample were captured, and PCR products having the predetermined sizes were obtained using the primer pairs BRCA1F1/BRCAARA and BRCA1F2/BRCA1R2, respectively.

These results indicate that MeLW1 and MeLW2 can effectively detect the methylated DNA fragments in a blood sample of a breast cancer patient without performing genomic DNA extraction in advance.

Summarizing the above test results, it is clear that the two DNA aptamers (i.e., MeLW1 and MeLW2) of the present disclosure exhibit high specificity and sensitivity in detection of a methylated DNA fragment in a biological sample by PCR, and thus, the two DNA aptamers are expected to be useful for evaluating the methylation of a gene of interest (e.g., a promoter of a tumor suppressor gene).

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A DNA aptamer specifically binding to a methylated DNA fragment, comprising a nucleotide sequence selected from the group consisting of SEQ ID NO:7 and SEQ ID NO:9.

2. A method for detecting a methylated DNA fragment in a sample, comprising:
mixing a DNA aptamer as claimed in claim 1 with the sample, so that the DNA aptamer binds to the methylated DNA fragment in the sample to form a DNA aptamer-methylated DNA fragment complex; and
detecting the DNA aptamer-methylated DNA fragment complex.

3. The method as claimed in claim 2, wherein the methylated DNA fragment is derived from a promoter of a tumor suppressor gene selected from the group consisting of a promoter of breast cancer gene 1 (BRCA1), a promoter of breast cancer gene 2 (BRCA2), a promoter of Ras association domain family 1A (RASSF1A) gene, a promoter of opioid binding protein/cell adhesion molecule-like (OPCML) protein gene, a promoter of p16INK4α gene, a promoter of Krüppel-like transcription factor 11 (KLF11) gene, a promoter of ADP-ribosylhydrolase (ADPRH) gene, a promoter of globoside alpha-1,3-N-acetylgalactosaminyltransferase 1 (GBGT1) gene, a promoter of PDZ and LIM domain protein 2 (PDLIM2) gene, and combinations thereof.

4. The method as claimed in claim 3, wherein the promoter of the tumor suppressor gene is selected from the group consisting of a promoter of BRCA1 and a promoter of BRCA2.

5. The method as claimed in claim 2, wherein detecting the DNA aptamer-methylated DNA fragment complex is conducted by subjecting the DNA aptamer-methylated DNA fragment complex to a DNA amplification reaction.

6. A method for selecting a DNA aptamer specifically binding to a methylated DNA fragment, comprising:
providing a library of candidate DNA aptamers;
incubating the library of candidate DNA aptamers with the methylated DNA fragment attached to a solid support at a temperature ranging from 25° C. to 37° C. and under at least one of the following conditions: a) in the presence of phosphate-buffered saline having a pH value ranging from pH 5.5 to pH 7.4; b) in the presence of 10 mM to 50 mM of lactic acid; and c) in the presence of blood, so that a DNA aptamer binds to the methylated DNA fragment; and
amplifying the DNA aptamer bound to the methylated DNA fragment.

7. The method as claimed in claim 6, wherein each candidate DNA aptamer is a single-stranded DNA having 20 to 80 random nucleotides.

8. The method as claimed in claim 6, wherein the DNA aptamer has a nucleotide sequence selected from the group consisting of SEQ ID NO:7 and SEQ ID NO:9.
